(19) United States
(12) Patent Application Publication
(10) Pub. No.: US 2005/0065134 A1
(43) Pub. Date: Mar. 24, 2005

(54) 2-aklylidene-19-nor-vitamin D derivatives for the treatment of anorexia or low bone mass in females exhibiting aggressive athletic behavior

(75) Inventor: David D. Thompson, Gales Ferry, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/944,368

(22) Filed: Sep. 16, 2004

Related U.S. Application Data
(60) Provisional application No. 60/504,510, filed on Sep. 19, 2003.

(51) Int. Cl. A61K 31/59

(52) U.S. Cl. 514/167

(57) ABSTRACT

The present invention relates to methods of treating anorexia or low bone mass in females exhibiting aggressive athletic behavior, the methods comprising administering to a patient in need thereof a 2-alkylidene-19-nor-vitamin D derivative. Particularly, the present invention relates to methods of treating anorexia or low bone mass in females exhibiting aggressive athletic behavior, the methods comprising administering to a patient in need thereof 2-methylene-19-nor-20(S)-1α,25-dihydroxyvitamin D₃.

### CROSS-REFERENCE FOR RELATED APPLICATION

This application claims the benefit from U.S. Provisional Application No. 60/504,510, filed on Sep. 19, 2003.

### FIELD OF THE INVENTION

The present invention relates to methods of treating anorexia or low bone mass in females exhibiting aggressive athletic behavior, the methods comprising administering to a patient in need thereof a 2-alkylidene-19-nor-vitamin D derivative. Particularly, the present invention relates to methods of treating anorexia or low bone mass in females exhibiting aggressive athletic behavior, the methods comprising administering to a patient in need thereof 2-methylene-19-nor-20(S)-1′,25-dihydroxyvitamin D₃.

### BACKGROUND OF THE INVENTION

Vitamin D is a general term that refers to a group of steroid molecules. The active form of vitamin D, which is called 1,25-dihydroxyvitamin D₃ (1,25-dihydroxycholecalciferol), is biosynthesized in humans by the conversion of 7-dehydrocholesterol to vitamin D₃ (cholecalciferol). This conversion takes place in the skin and requires UV radiation, which is typically from sunlight. Vitamin D₃ is then metabolized in the liver to 25-hydroxyvitamin D₃ (25-hydroxycholecalciferol), which is then further metabolized in the kidneys to the active form of vitamin D, 1,25-dihydroxvitamin D₃. 1,25-dihydroxyvitamin D₃ is then distributed throughout the body where it binds to intracellular vitamin D receptors.

The active form of vitamin D is a hormone that is known to be involved in mineral metabolism and bone growth and facilitates intestinal absorption of calcium.

Vitamin D analogs are disclosed in U.S. Pat. No. 5,843,928, issued Dec. 1, 1998. The compounds disclosed are 2-alkylidene-19-nor-vitamin D derivatives and are characterized by low intestinal calcium transport activity and high bone calcium mobilization activity when compared to 1,25-dihydroxyvitamin D₃.

In has been found that the 2-alkylidene-19-nor-vitamin D derivatives and particularly the compound 2-methylene-19-nor-20(S)-1α,25-dihydroxyvitamin D₃, (also known as 2MD) can be used in the treatment of anorexia or low bone mass in females exhibiting aggressive athletic behavior.

### SUMMARY OF THE INVENTION

The present invention provides methods of treating anorexia or low bone mass in females exhibiting aggressive athletic behavior, comprising administering to a patient in need thereof a therapeutically effective amount of 2-methylene-19-nor-20(S)-1α,25-dihydroxyvitamin D₃ or a pharmaceutically acceptable salt or prodrug thereof.

### DETAILED DESCRIPTION OF THE INVENTION

The present invention relates to the treatment of anorexia or low bone mass in females exhibiting aggressive athletic behavior using a 2-alkylidene-19-nor-vitamin D derivative. In a preferred embodiment, the present invention relates to a method of treating anorexia or low bone mass in females exhibiting aggressive athletic behavior using 2-methylene-19-nor-20(S)-1α,25-dihydroxyvitamin D₃. 2-Alkylidene-19-nor-vitamin D derivatives that can be used in the present invention are disclosed U.S. Pat. No. 5,843,928, which derivatives are characterized by the general formula I shown below: 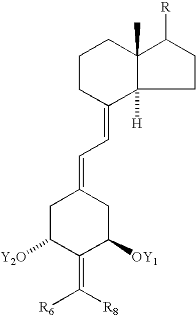
where Y₁ and Y₂, which may be the same or different, are each selected from the group consisting of hydrogen and a hydroxy-protecting group, R₆ and R₈, which may be the same or different, are each selected from the group consisting of hydrogen, alkyl, hydroxyalkyl and fluoroalkyl, or, when taken together represent the group —(CH₂)ₓ— where X is an integer from 2 to 5, and where the group R represents any of the typical side chains known for vitamin D type compounds.

More specifically R can represent a saturated or unsaturated hydrocarbon radical of 1 to 35 carbons, that may be straight-chain, branched or cyclic and that may contain one or more additional substituents, such as hydroxy- or protected-hydroxy groups, fluoro, carbonyl, ester, epoxy, amino or other heteroatomic groups. Preferred side chains of this type are represented by the structure below: 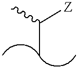
where the stereochemical center (corresponding to C-20 in steroid numbering) may have the R or S configuration (i.e., either the natural configuration about carbon 20 or the 20-epi configuration), and where Z is selected from Y, —OY, —CH₂OY, —C═CY and —CH═CHY, where the double bond may have the cis or trans geometry, and where Y is selected from hydrogen, methyl, —COR⁵ and a radical of the structure: 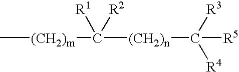
where m and n, independently, represent the integers from 0 to 5, where R¹ is selected from hydrogen, deuterium, hydroxy, protected hydroxy, fluoro, trifluoromethyl, and C₁₋₅-alkyl, which may be straight chain or branched and, optionally, bear a hydroxy or protected-hydroxy substituent, and where each of R², R³ and R⁴, independently, is selected from deuterium, deuteroalkyl, hydrogen, fluoro, trifluoromethyl and C₁₋₅ alkyl, which may be straight-chain or branched, and optionally, bear a hydroxy or protected-hydroxy substituent, and where R¹ and R², taken together, represent an oxo group, or an alkylidene group, ═CR²R³, or the group —(CH₂)ₚ—, where p is an integer from 2 to 5, and where R³ and R⁴, taken together, represent an oxo group, or the group —(CH₂)_{q}—, where q is an integer from 2 to 5, and where R⁵ represent hydrogen, hydroxy, protected hydroxy, or C₁₋₅ alkyl and wherein any of the CH-groups at positions 20, 22 or 23 in the side chain may be replaced by a nitrogen atom, or where any of the groups —CH(CH₃)—, —CH(R³)—, or —CH(R²)— at positions 20, 22 and 23, respectively, may be replaced by an oxygen or sulfur atom.

The wavy line to the methyl substituent at C-20 indicates that carbon 20 may have either the R or S configuration.

Specific important examples of side chains with natural 20R-configuration are the structures represented by formulas (a), (b), (c), (d) and (e) below, i.e., the side chain as it occurs in 25-hydroxyvitamin D₃ (a); vitamin D₃ (b); 25-hydroxyvitamin D₂ (c); vitamin D₂ (d); and the C-24 epimer of 25-hydroxyvitamin D₂ (e);

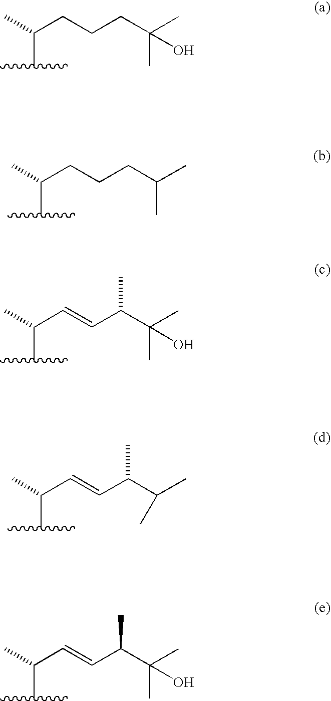

As used herein, the term “hydroxy-protecting group” signifies any group commonly used for the temporary protection of hydroxy functions, such as for example, alkoxycarbonyl, acyl, alkylsilyl or alkylarylsilyl groups (hereinafter referred to simply as “silyl” groups), and alkoxyalkyl groups. Alkoxycarbonyl protecting groups are alkyl-O—CO— groupings such as methoxycarbonyl, ethoxycarbonyl, propoxycarbonyl, isopropoxycarbonyl, butoxycarbonyl, isobutoxycarbonyl, tert-butoxycarbonyl, benzyloxycarbonyl or allyloxycarbonyl. The term “acyl” signifies an alkanoyl group of 1 to 6 carbons, in all of its isomeric forms, or a carboxyalkanoyl group of 1 to 6 carbons, such as an oxalyl, malonyl, succinyl, or glutaryl group, or an aromatic acyl group such as benzoyl, or a halo, nitro or alkyl substituted benzoyl group. The word “alkyl” as used in the description or the claims, denotes a straight-chain or branched alkyl radical of 1 to 10 carbons, in all its isomeric forms. Alkoxyalkyl protecting groups are groupings such as methoxymethyl, ethoxymethyl, methoxyethoxymethyl, or tetrahydrofuranyl and tetrahydropyranyl. Preferred silyl-protecting groups are trimethylsilyl, triethylsilyl, t-butyldimethylsilyl, dibutylmethylsilyl, diphenylmethylsilyl, phenyldimethylsilyl, diphenyl-t-butylsilyl and analogous alkylated silyl radicals. The term “aryl” specifies a phenyl-, or any alkyl-, nitro- or halo-substituted phenyl group.

A “protected hydroxy” group is a hydroxy group derivatized or protected by any of the above groups commonly used for the temporary or permanent protection of hydroxy functions, e.g., the silyl, alkoxyalkyl, acyl or alkoxycarbonyl groups, as previously defined. The terms “hydroxyalkyl”, “deuteroalkyl” and “fluoroalkyl” refer to any alkyl radical substituted by one or more hydroxy, deuterium or fluoro groups respectively.

It should be noted in this description that the term “24-homo” refers to the addition of one methylene group and the term “24-dihomo” refers to the addition of two methylene groups at the carbon 24 position in the side chain. Likewise, the term “trihomo” refers to the addition of three methylene groups. Also, the term “26,27-dimethyl” refers to the addition of a methyl group at the carbon 26 and 27 positions so that for example R³ and R⁴ are ethyl groups. Likewise, the term “26,27-diethyl” refers to the addition of an ethyl group at the 26 and 27 positions so that R³ and R⁴ are propyl groups.

In the following lists of compounds, the particular alkylidene substituent attached at the carbon 2 position should be added to the nomenclature. For example, if a methylene group is the alkylidene substituent, the term “2-methylene” should precede each of the named compounds. If an ethylene group is the alkylidene substituent, the term “2-ethylene” should precede each of the named compounds, and so on. In addition, if the methyl group attached at the carbon 20 position is in its epi or unnatural configuration, the term “20(S)” or “20-epi” should be included in each of the following named compounds. The named compounds could also be of the vitamin D₂ type if desired.

Specific and preferred examples of the 2-alkylidene-compounds of structure I when the side chain is unsaturated are:
19-nor-24-homo-1,25-dihydroxy-22-dehydrovitamin D₃;
19-nor-24-dihomo-1,25-dihydroxy-22-dehydrovitamin D₃;
19-nor-24-trihomo-1,25-dihydroxy-22-dehydrovitamin D₃;
19-nor-26,27-dimethyl-24-homo-1,25-dihydroxy-22-dehydrovitamin D₃;
19-nor-26,27-dimethyl-24-dihomo-1,25-dihydroxy-22-dehydrovitamin D₃;
19-nor-26,27-dimethyl-24-trihomo-1,25-dihydroxy-22-dehydrovitamin D₃;
19-nor-26,27-diethyl-24-homo-1,25-dihydroxy-22-dehydrovitamin D₃;
19-nor-26,27-diethyl-24-dihomo-1,25-dihydroxy-22-dehydrovitamin D₃;
19-nor-26,27-diethyl,24-trihomo-1,25-dihydroxy-22-dehydrovitamin D₃;
19-nor-26,27-dipropyl-24-homo-1,25-dihydroxy-22-dehydrovitamin D₃;
19-nor-26,27-dipropyl-24-dihomo-1,25-dihydroxy-22-dehydrovitamin D₃; and
19-nor-26,27-dipropyl-24-trihomo-1,25-dihydroxy-22-dehydrovitamin D₃.

Specific and preferred examples of the 2-alkylidene-compounds of structure I when the side chain is saturated are:
19-nor-24-homo-1,25-dihydroxyvitamin D₃;
19-nor-24-dihomo-1,25-dihydroxyvitamin D₃;
19-nor-24-trihomo-1,25-dihydroxyvitamin D₃;
19-nor-26,26-dimethyl-24-homo-1,25-dihydroxyvitamin D₃;
19-nor-26,27-dimethyl-24-dihomo-1,25-dihydroxyvitamin D₃;
19-nor-26,27-dimethyl-24-trihomo-1,25-dihydroxyvitamin D₃;
19-nor-26,27-diethyl-24-homo-1,25-dihydroxyvitamin D₃;
19-nor-26,27-diethyl-24-dihomo-1,25-dihydroxyvitamin D₃;
19-nor-26,27-diethyl-24-trihomo-1,25-dihydroxyvitamin D₃;
19-nor-26,27-dipropyl-24-homo-1,25-dihydroxyvitamin D₃;
19-nor-26,27-dipropyl-24-dihomo-1,25-dihydroxyvitamin D₃; and
19-nor-26,27-dipropyl-24-trihomo-1,25-dihydroxyvitamin D₃.

Anorexia is a disease that has the following characterisitcs: refusal to maintain body weight at or above a minimally normal weight for age and height (e.g., weight loss leading to maintenance of body weight less than 85% of that expected; or failure to make expected weight gain during period of growth, leading to body weight less than 85% of that expected);intense fear of gaining weight or becoming fat, even though underweight; and disturbance in the way in which one's body weight or shape is experienced, undue influence of body weight or shape on self-evaluation, or denial of the seriousness of the current low body weight. The compounds and combinations of the present invention can be used to treat anorexia and can be used to treat bone loss associated with anorexia.

Another condition that can be treated using the compounds and combinations of the present invention is bone loss associated with aggressive athletic behavior, particularly in women. Aggressive participation in exercise, athletics or sports can result in bone loss, which is usually accompanied in women by ammenorhea. Men who also exhibit aggressive athletic behavior also exhibit bone loss.

The present invention is also concerned with pharmaceutical compositions for the treatment of anorexia or low bone mass in females exhibiting aggressive athletic behavior comprising administering to a patient in need thereof a 2-alkylidene-19-nor-vitamin D derivative, such as a compound of Formula I, and a carrier, solvent, diluent and the like.

It is noted that when compounds are discussed herein, it is contemplated that the compounds may be administered to a patient as a pharmaceutically acceptable salt, prodrug, or a salt of a prodrug. All such variations are intended to be included in the invention.

The term “patient in need thereof” means humans and other animals who have or are at risk of having anorexia or low bone mass in females exhibiting aggressive athletic behavior.

The term “treating”, “treat” or “treatment” as used herein includes preventative (e.g., prophylactic), palliative and curative treatment.

By “pharmaceutically acceptable” it is meant the carrier, diluent, excipients, and/or salts or prodrugs must be compatible with the other ingredients of the formulation, and not deleterious to the patient.

The term “prodrug” means a compound that is transformed in vivo to yield a compound of the present invention. The transformation may occur by various mechanisms, such as through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, when a compound of the present invention contains a carboxylic acid functional group, a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, when a compound of the present invention comprises an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

When a compound of the present invention comprises an amine functional group, a prodrug can be formed by the replacement of a hydrogen atom in the amine group with a group such as R^{X}-carbonyl, R^{X}O-carbonyl, NR^{X}R^{X}′-carbonyl where R^{X} and R^{X}′ are each independently (C₁-C₁₀)alkyl, (C₃-C₇)cycloalkyl, benzyl, or R^{X}-carbonyl is a natural α-aminoacyl or natural α-aminoacyl-natural α-aminoacyl, —C(OH)C(O)OY^{X} wherein Y^{X} is H, (C₁-C₆)alkyl or benzyl), —C(OY^{X0})Y^{X1} wherein Y^{X0} is (C₁-C₄) alkyl and Y^{X1} is (C₁-C₆)alkyl, carboxy(C₁-C₆)alkyl, amino(C₁-C₄)alkyl or mono-N- or di-N,N-(C₁-C₆)alkylaminoalkyl, —C(Y^{X2})Y^{X3} wherein Y^{X2} is hydrogen or methyl and Y^{X3} is mono-N- or di-N,N-(C₁-C₆)alkylamino, morpholino, piperidin-1-yl or pyrrolidin-1-yl.

The expression “pharmaceutically acceptable salt” refers to nontoxic anionic salts containing anions such as (but not limited to) chloride, bromide, iodide, sulfate, bisulfate, phosphate, acetate, maleate, fumarate, oxalate, lactate, tartrate, citrate, gluconate, methanesulfonate and 4-toluene-sulfonate. The expression also refers to nontoxic cationic salts such as (but not limited to) sodium, potassium, calcium, magnesium, ammonium or protonated benzathine (N,N′-dibenzylethylenediamine), choline, ethanolamine, diethanolamine, ethylenediamine, meglamine (N-methyl-glucamine), benethamine (N-benzylphenethylamine), piperazine or tromethamine (2-amino-2-hydroxymethyl-1,3-propanediol).

It will be recognized that the compounds of this invention can exist in radiolabelled form, i.e., said compounds may contain one or more atoms containing an atomic mass or mass number different from the atomic mass or mass number ordinarily found in nature. Radioisotopes of hydrogen, carbon, phosphorous, fluorine and chlorine include ³H, ¹⁴C, ³²P, ³⁵S, ¹⁸F and ³⁶Cl, respectively. Compounds of this invention which contain those radioisotopes and/or other radioisotopes of other atoms are within the scope of this invention. Tritiated, i.e., ³H, and carbon-14, i.e., ¹⁴C, radioisotopes are particularly preferred for their ease of preparation and detectability. Radiolabelled compounds of this invention can generally be prepared by methods well known to those skilled in the art. Conveniently, such radiolabelled compounds can be prepared by carrying out the procedures disclosed herein except substituting a readily available radiolabelled reagent for a non-radiolabelled reagent.

It will be recognized by persons of ordinary skill in the art that some of the compounds of this invention have at least one asymmetric carbon atom and therefore are enantiomers or diastereomers. Diasteromeric mixtures can be separated into their individual diastereomers on the basis of their physicochemical differences by methods known per se as, for example, chromatography and/or fractional crystallization. Enantiomers can be separated by converting the enantiomeric mixture into a diasteromeric mixture by reaction with an appropriate optically active compound (e.g., alcohol), separating the diastereomers and converting (e.g., hydrolyzing, including both chemical hydrolysis methods and microbial lipase hydrolysis methods, e.g., enzyme catalyzed hydrolysis) the individual diastereomers to the corresponding pure enantiomers. All such isomers, including diastereomers, enantiomers and mixtures thereof are considered as part of this invention. Also, some of the compounds of this invention are atropisomers (e.g., substituted biaryls) and are considered as part of this invention.

In addition, when the compounds of this invention, including the compounds of Formula I, form hydrates or solvates, they are also within the scope of the invention.

Administration of the compounds of this invention can be via any method that delivers a compound of this invention systemically and/or locally. These methods include oral, parenteral, and intraduodenal routes, etc. Generally, the compounds of this invention are administered orally, but parenteral administration (e.g., intravenous, intramuscular, transdermal, subcutaneous, rectal or intramedullary) may be utilized, for example, where oral administration is inappropriate for the target or where the patient is unable to ingest the drug.

The compounds of this invention may also be applied locally to a site in or on a patient in a suitable carrier or diluent.

2MD and other 2-alkylidene-19-nor-vitamin D derivatives of the present invention can be administered to a human patient in the range of about 0.01 μg/day to about 10 μg/day. A preferred dosage range is about 0.05 μg/day to about 1 μg/day and a more preferred dosage range is about 0.1 μg/day to about 0.4 μg/day. The amount and timing of administration will, of course, be dependent on the subject being treated, on the severity of the affliction, on the manner of administration and on the judgment of the prescribing physician. Thus, because of patient to patient variability, the dosages given herein are guidelines and the physician may titrate doses of the drug to achieve the treatment that the physician considers appropriate for the patient. In considering the degree of treatment desired, the physician must balance a variety of factors such as age of the patient, presence of preexisting disease, as well as presence of other diseases. The dose may be given once a day or more than once a day and may be given in a sustained release or controlled release formulation. It is also possible to administer the compounds using a combination of an immediate release and a controlled release and/or sustained release formulation.

The administration of 2MD or other 2-alkylidene-19-nor-vitamin D derivative can be according to any continuous or intermittent dosing schedule. Once a day, multiple times a day, once a week, multiple times a week, once every two weeks, multiple times every two weeks, once a month, multiple times a month, once every two months, once every three months, once every six months and once a year dosing are non-limiting examples of dosing schedules for 2MD or another 2-alkylidene-19-nor-vitamin D derivative.

The compounds of the present invention are generally administered in the form of a pharmaceutical composition comprising at least one of the compounds of this invention together with a pharmaceutically acceptable vehicle or diluent. Thus, the compounds of this invention can be administered in any conventional oral, parenteral, rectal or transdermal dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof. One example of an acceptable formulation for 2MD and other 2-alkylidene-19-nor-vitamin D derivatives is a soft gelatin capsule containing neobe oil in which the 2MD or other 2-alkylidene-19-nor-vitamin D derivative has been dissolved. Other suitable formulations will be apparent to those skilled in the art.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easton, Pa., 19th Edition (1995).

Advantageously, the present invention also provides kits for use by a consumer to treat anorexia or low bone mass in females exhibiting aggressive athletic behavior. The kits comprise a) a pharmaceutical composition comprising a 2-alkylidene-19-nor-vitamin D derivative, and particularly, the compound 2-methylene-19-nor-20(S)-1α,25-dihydroxyvitamin D₃, and a pharmaceutically acceptable carrier, vehicle or diluent; and b) instructions describing a method of using the pharmaceutical composition to treat anorexia or low bone mass in females exhibiting aggressive athletic behavior.

A “kit” as used in the instant application includes a container for containing the pharmaceutical compositions and may also include divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a written memory aid, where the written memory aid is of the type containing information and/or instructions for the physician, pharmacist or patient, e.g., in the form of numbers next to the tablets or capsules whereby the numbers correspond with the days of the regimen which the tablets or capsules so specified should be ingested or a card which contains the same type of information. Another example of such a memory aid is a calendar printed on the card e.g., as follows “First Week, Monday, Tuesday,” . . . et . . . “Second Week, Monday, Tuesday, . . . ” etc. Other variations of memory aids will be readily apparent. A “daily dose” can be a single tablet or capsule or several tablets or capsules to be taken on a given day.

Another specific embodiment of a kit is a dispenser designed to dispense the daily doses one at a time. Preferably, the dispenser is equipped with a memory-aid, so as to further facilitate compliance with the regimen. An example of such a memory-aid is a mechanical counter which indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The preparation of 1α-hydroxy-2-alkyl-19-nor-vitamin D compounds, particularly 1α-hydroxy-2-methyl-19-nor-vitamin D compounds, having the basic structure I can be accomplished by a common general method, i.e., the condensation of a bicyclic Windaus-Grundmann type ketone II with the allylic phosphine oxide III to the corresponding 2-methylene-19-nor-vitamin D analogs IV followed by deprotection at C-1 and C-3 in the latter compounds:

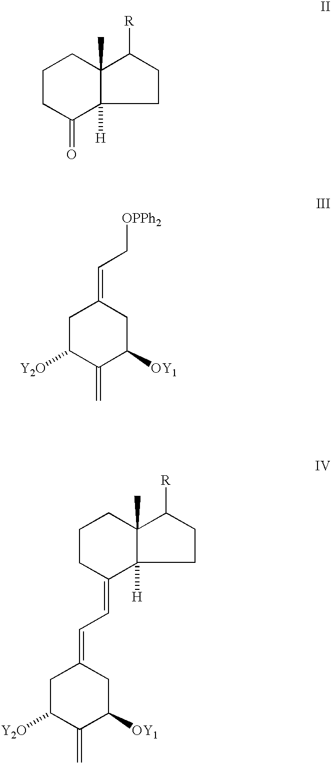

In the structures II, III, and IV groups Y₁ and Y₂ and R represent groups defined above; Y₁ and Y₂ are preferably hydroxy-protecting groups, it being also understood that any functionalities in R that might be sensitive, or that interfere with the condensation reaction, be suitably protected as is well-known in the art. The process shown above represents an application of the convergent synthesis concept, which has been applied effectively for the preparation of vitamin D compounds [e.g., Lythgoe et al., J. Chem. Soc. Perkin Trans. 1, 590 (1978); Lythgoe, Chem. Soc. Rev. 9, 449 (1983); Toh et al., J. Org. Chem. 48, 1414 (1983); Baggiolini et al., J. Org. Chem. 51, 3098 (1986); Sardina et al,. J. Org. Chem. 51, 1264 (1986); J. Org. Chem. 51, 1269 (1986); DeLuca et al., U.S. Pat. No. 5,086,191; DeLuca et al., U.S. Pat. No. 5,536,713].

Hydrindanones of the general structure II are known, or can be prepared by known methods. Specific important examples of such known bicyclic ketones are the structures with the side chains (a), (b), (c) and (d) described above, i.e., 25-hydroxy Grundmann's ketone (f) [Baggiolini et al., J. Org. Chem. 51, 3098 (1986)]; Grundmann's ketone (g) [Inhoffen et al., Chem. Ber. 90, 664 (1957)]; 25-hydroxy Windaus ketone (h) [Baggiolini et al., J. Org. Chem. 51, 3098 (1986)] and Windaus ketone (i) [Windaus et al., Ann., 524, 297 (1936)]:

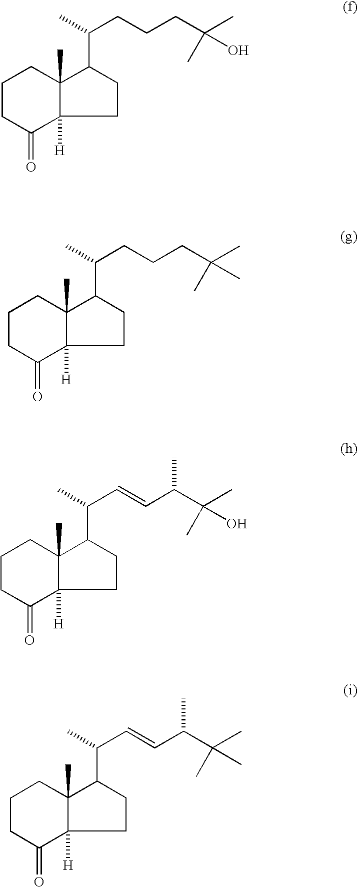

For the preparation of the required phosphine oxides of general structure III, a new synthetic route has been developed starting from methyl quinicate derivative 1, easily obtained from commercial (1R,3R,4S,5R)-(−)-quinic acid as described by Perlman et al., Tetrahedron Lett. 32, 7663 (1991) and DeLuca et al., U.S. Pat. No. 5,086,191. The overall process of transformation of the starting methyl ester 1 into the desired A-ring synthons, is summarized by Scheme I. Thus, the secondary 4-hydroxyl group of 1 was oxidized with RuO₄ (a catalytic method with RuCl₃ and NalO₄ as co-oxidant). Use of such a strong oxidant was necessary for an effective oxidation process of this very hindered hydroxyl. However, other more commonly used oxidants can also be applied (e.g., pyridinium dichromate), although the reactions usually require much longer time for completion. The second step of the synthesis comprises the Wittig reaction of the sterically hindered 4-keto compound 2 with the ylide prepared from methyltriphenylphosphonium bromide and n-butyllithium. Other bases can be also used for the generation of the reactive methylenephosphorane, like t-BuOK, NaNH₂, NaH, K/HMPT, NaN(TMS)₂, etc. For the preparation of the 4-methylene compound 3 some described modifications of the Wiltig process can be used, e.g., reaction of 2 with activated methylenetriphenylphosphorane [Corey et al., Tetrahedron Lett. 26, 555 (1985)]. Alternatively, other methods widely used for methylenation of unreactive ketones can be applied, e.g., Wittig-Horner reaction with the PO-ylid obtained from methyldiphenylphosphine oxide upon deprotonation with n-butyllithium [Schosse et al., Chimia 30, 197 (1976)], or reaction of ketone with sodium methylsulfinate [Corey et al., J. Org. Chem. 28, 1128 (1963)] and potassium methylsulfinate [Greene et al., Tetrahedron Lett. 3755 (1976)]. Reduction of the ester 3 with lithium aluminum hydride or other suitable reducing agent (e.g., DIBALH) provided the diol 4 which was subsequently oxidized by sodium periodate to the cyclohexanone derivative 5. The next step of the process comprises the Peterson reaction of the ketone 5 with methyl(trimethylsilyl)acetate. The resulting allylic ester 6 was treated with diisobutylaluminum hydride and the formed allylic alcohol 7 was in turn transformed to the desired A-ring phosphine oxide 8. Conversion of 7 to 8 involved 3 steps, namely, in situ tosylation with n-butyllithium and p-toluenesulfonyl chloride, followed by reaction with diphenylphosphine lithium salt and oxidation with hydrogen peroxide.

Several 2-methylene-19-nor-vitamin D compounds of the general structure IV may be synthesized using the A-ring synthon 8 and the appropriate Windaus-Grundmann ketone II having the desired side chain structure. Thus, for example, Wittig-Horner coupling of the lithium phosphinoxy carbanion generated from 8 and n-butyllithium with the protected 25-hydroxy Grundmann's ketone 9 prepared according to published procedure [Sicinski et al., J. Med. Chem. 37, 3730 (1994)] gave the expected protected vitamin compound 10. This, after deprotection with AG 50W-X4 cation exchange resin afforded 1α,25-dihydroxy-2-methylene-19-nor-vitamin D₃ (11).

The C-20 epimerization was accomplished by the analogous coupling of the phosphine oxide 8 with protected (20S)-25-hydroxy Grundmann's ketone 13 (Scheme II) and provided 19-nor-vitamin 14 which after hydrolysis of the hydroxy-protecting groups gave (20S)-1α,25-dihydroxy-2-methylene-19-nor-vitamin D₃ (15). As noted above, other 2-methylene-19-nor-vitamin D analogs may be synthesized by the method disclosed herein. For example, 1α-hydroxy-2-methylene-19-nor-vitamin D₃ can be obtained by providing the Grundmann's ketone (g).

All documents cited in this application, including patents and patent applications, are hereby incorporated by reference. The examples presented below are intended to illustrate particular embodiments of the invention and are not intended to limit the invention, including the claims, in any manner.

### EXAMPLES

The following abbreviations are used in this application.

| | |
|---|---|
| NMR | nuclear magnetic resonance |
| mp | melting point |
| H | hydrogen |
| h | hour(s) |
| min | minutes |
| t-Bu | tert-butyl |
| THF | tetrahydrofuran |
| n-BuLi | n-butyl lithium |
| MS | mass spectra |
| HPLC | high pressure liquid chromatography |
| SEM | standard error measurement |
| Ph | phenyl |
| Me | methyl |
| Et | ethyl |
| DIBALH | diisobutylaluminum hydride |
| LDA | lithium diisopropylamide |

The preparation of compounds of Formula I were set forth in U.S. Pat. No. 5,843,928 as follows:

In these examples, specific products identified by Arabic numerals (e.g., 1, 2, 3, etc.) refer to the specific structures so identified in the preceding description and in Scheme I and Scheme II.

### Example 1

Preparation of 1α,25-dihydroxy-2-methylene-19-nor-vitamin D₃ (11)

Referring first to Scheme I the starting methyl quinicate derivative 1 was obtained from commercial (−)-quinic acid as described previously [Perlman et al., Tetrahedron Lett. 32, 7663 (1991) and DeLuca et al., U.S. Pat. No. 5,086,191]. 1:mp. 82°-82.5° C. (from hexane), ¹H NMR(CDCl₃) δ0.098, 0.110, 0.142, and 0.159 (each 3H, each s, 4×SiCH₃), 0.896 and 0.911 (9H and 9H, each s, 2×Si-t-Bu), 1.820 (1H, dd, J=13.1, 10.3 Hz), 2.02 (1H, ddd, J=14.3, 4.3, 2.4 Hz), 2.09 (1H, dd, J=14.3, 2.8 Hz), 2.19 (1H, ddd, J=13.1, 4.4, 2.4 Hz), 2.31 (1H, d, J=2.8 Hz, OH), 3.42 (1H, m; after D₂O dd, J=8.6, 2.6 Hz), 3.77 (3H,s), 4.12 (1H,m), 4.37 (1H, m), 4.53 (1H,br s, OH).

(a) Oxidation of 4-hydroxy group in Methyl Quinicate Derivative 1

(3R,5R)-3,5-Bis[(tert-butyldimethylsilyl)oxy]-1-hydroxy-4-oxocyclohexanecarboxylic Acid Methyl Ester (2). To a stirred mixture of ruthenium (III) chloride hydrate (434 mg, 2.1 mmol) and sodium periodate (10.8 g, 50.6 mmol) in water (42 mL) was added a solution of methyl quinicate 1 (6.09 g, 14 mmol) in CCl₄/CH₃CN (1:1, 64 mL). Vigorous stirring was continued for 8 h. Few drops of 2-propanol were added, the mixture was poured into water and extracted with chloroform. The organic extracts were combined, washed with water, dried (MgSO₄) and evaporated to give a dark oily residue (ca. 5 g) which was purified by flash chromatography. Elution with hexane/ethyl acetate (8:2) gave pure, oily 4-ketone 2 (3.4 g, 56%): ¹H NMR (CDCl₃) δ0.054, 0.091, 0.127, and 0.132 (each 3H, each s, 4×SiCH₃), 0.908 and 0.913 (9H and 9H, each s, 2×Si-t-Bu), 2.22 (1H, dd, J=13.2, 11.7 Hz), 2.28 (1 H, ˜dt J=14.9, 3.6 Hz), 2.37 (1 H, dd, J=14.9, 3.2 Hz), 2.55 (1H, ddd, J=13.2, 6.4, 3.4 Hz), 3.79 (3H,s), 4.41 (1H, t, J-3.5 Hz), 4.64 (1H, s, OH), 5.04 (1H, dd, J=1 1.7, 6.4 Hz); MS m/z (relative intensity) no M+, 375 (M+-t-Bu, 32), 357 (M+-t-Bu—H₂O, 47), 243 (31), 225 (57), 73 (100).

(b) Wittig Reaction of the 4-ketone 2

(3R,5R)-3,5-Bis[(tert-butyldimethylsilyl)oxy]-1-hydroxy-4-methylenecyclohexanecarboxylic Acid Methyl Ester (3). To the methyltriphenylphoshonium bromide (2.813 g, 7.88 mmol) in anhydrous THF (32 mL) at 08 C. was added dropwise n-BuLi (2.5M in hexanes, 6.0 mL, 15 mmol) under argon with stirring. Another portion of MePh₃P⁺Br⁻ (2.813 g, 7.88 mmol) was then added and the solution was stirred at 0° C. for 10 min. and at room temperature for 40 min. The orange-red mixture was again cooled to 0° C. and a solution of 4-ketone 2 (1.558 g, 3.6 mmol) in anhydrous THF (16+2 mL) was syphoned to reaction flask during 20 min. The reaction mixture was stirred at 0° C. for 1 h. and at room temperature for 3 h. The mixture was then carefully poured into brine cont. 1% HCl and extracted with ethyl acetate and benzene. The combined organic extracts were washed with diluted NaHCO₃ and brine, dried (MgSO₄) and evaporated to give an orange oily residue (ca. 2.6 g) which was purified by flash chromatography. Elution with hexane/ethyl acetate (9:1) gave pure 4-methylene compound 3 as a colorless oil (368 mg, 24%): ¹H NMR (CDCl₃) δ0.078, 0.083, 0.092, and 0.115 (each 3H, each s, 4×SiCH₃), 0.889 and 0.920 (9H and 9H, each s, 2×Si-t-Bu), 1.811 (1 H, dd, J=12.6, 11.2 Hz), 2.10 (2H, m), 2.31 (1H, dd, J=12.6, 5.1 Hz), 3.76 (3H, s), 4.69 (1H, t, J=3.1 Hz), 4.78 (1H, m), 4.96 (2H, m; after D₂O 1H, br s), 5.17 (1H, t, J=1.9 Hz); MS m/z (relative intensity) no M+, 373 (M+-t-Bu, 57), 355 (M+-t-Bu -H₂O, 13), 341 (19), 313 (25), 241 (33), 223 (37), 209 (56), 73 (100).

(c) Reduction of Ester Group in the 4-methylene Compound 3

[(3R,5R)-3,5-Bis[(tert-butyldimethylsilyl)oxy]-1-hydroxy-4-methylenecyclohexyl]methanol (4). (i) To a stirred solution of the ester 3 (90 mg, 0.21 mmol) in anhydrous THF (8 mL) lithium aluminum hydride (60 mg, 1.6 mmol) was added at 0° C. under argon. The cooling bath was removed after 1 h. and the stirring was continued at 6° C. for 12 h. and at room temperature for 6 h. The excess of the reagent was decomposed with saturated aq. Na₂SO₄, and the mixture was extracted with ethyl acetate and ether, dried (MgSO₄) and evaporated. Flash chromatography of the residue with hexane/ethyl acetate (9:1) afforded unreacted substrate (12 mg) and a pure, crystalline diol 4 (35 mg, 48% based on recovered ester 3): ¹H NMR (CDCl₃+D₂O) δ0.079, 0.091, 0.100, and 0.121 (each 3H, each s, 4×SiCH₃), 0.895 and 0.927 (9H and 9H, each s, 2×Si-t-Bu), 1.339 (1H, t, J˜12 Hz), 1.510 (1H, dd, J=14.3, 2.7 Hz), 2.10 (2H, m), 3.29 and 3.40 (1H and 1H, each d, J=11.0 Hz), 4.66 (1H, t, J˜2.8 Hz), 4.78 (1H, m), 4.92 (1H, t, J=1.7 Hz), 5.13 (1H, t, J=2.0 Hz); MS m/z (relative intensity) no M+, 345 (M+-t-Bu, 8), 327 (M+-t-Bu-H₂0, 22), 213 (28), 195 (11), 73 (100).

(ii) Diisobutylaluminum hydride (1.5M in toluene, 2.0 mL, 3 mmol) was added to a solution of the ester 3 (215 mg, 0.5 mmol) in anhydrous ether (3 mL) at −78° C. under argon. The mixture was stirred at −78° C. for 3 h. and at −24° C. for 1.5 h., diluted with ether (10 mL) and quenched by the slow addition of 2N potassium sodium tartrate. The solution was warmed to room temperature and stirred for 15 min., the poured into brine and extracted with ethyl acetate and ether. The organic extracts were combined, washed with diluted (ca. 1%) HCl, and brine, dried (MgSO₄) and evaporated. The crystalline residue was purified by flash chromatography. Elution with hexane/ethyl acetate (9:1) gave crystalline diol 4 (43 mg, 24%).

(d) Cleavage of the Vicinal Diol 4

(3R,5R)-3,5-Bis[(tert-butyldimethylsilyl)oxy]-4-methylenecyclohexanone (5). Sodium periodate saturated water (2.2 mL) was added to a solution of the diol 4 (146 mg, 0.36 mmol) in methanol (9 mL) at 0° C. The solution was stirred at 0° C. for 1 h., poured into brine and extracted with ether and benzene. The organic extracts were combined, washed with brine, dried (MgSO₄) and evaporated. An oily residue was dissolved in hexane (1 mL) and applied on a silica Sep-Pak cartridge. Pure 4-methylenecyclohexanone derivative 5 (110 mg, 82%) was eluted with hexane/ethyl acetate (95:5) as a colorless oil: ¹H NMR (CDCl₃) δ0.050 and 0.069 (6H and 6H, each s, 4×SiCH₃), 0.881 (18H, s, 2×Si-t-Bu), 2.45 (2H, ddd, J=14.2, 6.9, 1.4 Hz), 2.64 (2H, ddd, J=14.2, 4.6, 1.4 Hz), 4.69 (2H, dd, J=6.9, 4.6 Hz), 5.16 (2H, s); MS M/z (relative intensity) no M+, 355 (M+-Me, 3), 313 (M+-t-Bu, 100), 73 (76).

(e) Preparation of the Allylic Ester 6

[(3′R,5′R)-3′,5′-Bis[(tert-butyldimethylsilyl)oxy]4′-methylenecyclohexylidene]acetic Acid Methyl Ester (6). To a solution of diisopropylamine (37 μL, 0.28 mmol) in anhydrous THF (200 μL) was added n-BuLi (2.5M in hexanes, 113 μL, 0.28 mmol) under argon at −78° C. with stirring, and methyl(trimethylsilyl)acetate (46 μL, 0.28 mmol) was then added. After 15 min., the keto compound 5 (49 mg, 0.132 mmol) in anhydrous THF (200+80 μL) was added dropwise. The solution was stirred at −78° C. for 2 h. and the reaction mixture was quenched with saturated NH₄Cl, poured into brine and extracted with ether and benzene. The combined organic extracts were washed with brine, dried (MgSO₄) and evaporated. The residue was dissolved in hexane (1 mL) and applied on a silica Sep-Pak cartridge. Elution with hexane and hexane/ethyl acetate (98:2) gave a pure allylic ester 6 (50 mg, 89%) as a colorless oil: ¹H NMR (CDCl₃) δ0.039, 0.064, and 0.076 (6H, 3H, and 3H, each s, 4×SiCH₃), 0.864 and 0.884 (9H and 9H, each s, 2×Si-t-Bu), 2.26 (1H, dd, J=12.8, 7.4 Hz), 2.47 (1H, dd, J=12.8, 4.2 Hz), 2.98 (1H, dd, J=13.3, 4.0 Hz), 3.06 (1H, dd, J=13.3, 6.6 Hz), 3.69 (3H, s), 4.48 (2H, m), 4.99 (2H, s), 5.74 (1H, s); MS m/z (relative intensity) 426 (M+, 2), 411 (M+-Me, 4), 369 (M+-t-Bu, 100), 263 (69).

(f) Reduction of the Allylic Ester 6

2-[(3′R,5′R)-3′,5′-Bis[(tert-butyidimethylsilyl)oxy]-4′-methylenecyclohexylidene]ethanol (7). Diisobutylaluminum hydride (1.5M in toluene, 1.6 mL, 2.4 mmol) was slowly added to a stirred solution of the allylic ester 6 (143 mg, 0.33 mmol) in toluene/methylene chloride (2:1, 5.7 mL) at −788 C. under argon. Stirring was continued as −78° C. for 1 h. and at −46° C. (cyclohexanone/dry ice bath) for 25 min. The mixture was quenched by the slow addition of potassium sodium tartrate (2N, 3 mL), aq. HCl (2N, 3 mL) and H₂O (12 mL), and then diluted with methylene chloride (12 mL) and extracted with ether and benzene. The organic extracts were combined, washed with diluted (ca. 1 %) HCl, and brine, dried (MgSO₄) and evaporated. The residue was purified by flash chromatography. Elution with hexane/ethyl acetate (9:1) gave crystalline allylic alcohol 7 (130 mg, 97%): ¹H NMR (CDCl₃) δ0.038, 0.050, and 0.075 (3H, 3H, and 6H, each s, 4×SiCH₃), 0.876 and 0.904 (9H and 9H, each s, 2×Si-t-Bu), 2.12 (1H, dd J=12.3, 8.8 Hz), 2.23 (1H, dd, J=13.3, 2.7 Hz), 2.45 (1H, dd, J=12.3, 4.8 Hz), 2.51 (1H, dd, J=13.3, 5.4 Hz), 4.04 (1H, m; after D₂O dd, J=12.0, 7.0 Hz), 4.17 (1H, m; after D₂O dd, J=12.0, 7.4 Hz), 4.38 (1 H, m), 4.49 (1 H, m), 4.95 (1 H, br s), 5.05 (1 H, t, J=1.7 Hz), 5.69 (1H, ˜t, J=7.2 Hz); MS m/z (relative intensity) 398 (M+, 2), 383 (M+-Me, 2), 365 (M+-Me-H₂O, 4), 341 (M+-t-Bu, 78), 323 (M+-t-Bu-H₂O, 10), 73 (100).

(g) Conversion of the Allylic Alcohol 7 Into Phosphine Oxide 8

[2-[(3′R,5′R)-3′,5′-Bis[(tert-butyldimethylsilyl)oxy]4′-methylenecyclohexylidene]ethyl]diphenylphosphine Oxide (8). To the allylic alcohol 7 (105 mg, 0.263 mmol) in anhydrous THF (2.4 mL) was added n-BuLi (2.5M in hexanes, 105 μL, 0.263 mmol) under argon at 0° C. Freshly recrystallized tosyl chloride (50.4 mg, 0.264 mmol) was dissolved in anhydrous THF (480 μL) and added to the allylic alcohol-BuLi solution. The mixture was stirred at 0° C. for 5 min. and set aside at 0° C. In another dry flask with air replaced by argon, n-BuLi (2.5M in hexanes, 210 μL, 0.525 mmol) was added to Ph₂PH (93 μL, 0.534 mmol in anhydrous THF (750 μL) at 0° C. with stirring. The red solution was siphoned under argon pressure to the solution of tosylate until the orange color persisted (ca. ½ of the solution was added). The resulting mixture was stirred an additional 30 min. at 0° C., and quenched by addition of H₂O (30 μL). Solvents were evaporated under reduced pressure and the residue was redissolved in methylene chloride (2.4 mL) and stirred with 10% H₂O₂ at 0° C. for 1 h. The organic layer was separated, washed with cold aq. sodium sulfite and H₂O, dried (MgSO₄) and evaporated. The residue was subject to flash chromatography. Elution with benzene/ethyl acetate (6:4) gave semicrystalline phosphine oxide 8 (134 mg, 87%): ¹H NMR (CDCl₃) δ0.002, 0.011 and 0.019 (3H, 3H, and 6H, each s, 4×SiCH₃), 0.855 and 0.860 (9H and 9H, each s, 2×Si-t-Bu), 2.0-2.1 (3H, br m), 2.34 (1 H, m), 3.08 (1 H, m), 3.19 (1 H, m), 4.34 (2H, m), 4.90 and 4.94 (1H and 1H, each s,), 5.35 (1H, ˜q, J=7.4 Hz), 7.46 (4H, m), 7.52 (2H, m), 7.72 (4H, m); MS m/z (relative intensity) no M+, 581 (M+-1, 1), 567 (M+-Me, 3) 525 (M+-t-Bu, 100), 450 (10), 393 (48).

(h) Wittig-Horner Coupling of Protected 25-hydroxy Grundmann's Ketone 9 with the Phosphine Oxide 8

1α,25-Dihydroxy-2-methylene-19-nor-vitamin D₃ (11). To a solution of phosphine oxide 8 (33.1 mg, 56.8 μmol) in anhydrous THF (450 μL) at 0° C. was slowly added n-BuLi (2.5M in hexanes, 23 μL, 57.5 μmol) under argon with stirring. The solution turned deep orange. The mixture was cooled to −78° C. and a precooled (−78° C.) solution of protected hydroxy ketone 9 (9.0 mg, 22.8 μmol), prepared according to published procedure [Sicinski et al., J. Med. Chem. 37, 3730 (1994)], in anhydrous THF (200+100 μL) was slowly added. The mixture was stirred under argon at −78° C. for 1 h. and at 0° C. for 18 h. Ethyl acetate was added, and the organic phase was washed with brine, dried (MgSO₄) and evaporated. The residue was dissolved in hexane and applied on a silica Sep-Pak cartridge, and washed with hexane/ethyl acetate (99:1, 20 mL) to give 19-nor-vitamin derivative 10 (13.5 mg, 78%). The Sep-Pak was then washed with hexane/ethyl acetate (96:4), 10 mL) to recover some unchanged C,D-ring ketone 9 (2 mg), and with ethyl acetate (10 mL) to recover diphenylphosphine oxide (20 mg). For analytical purpose a sample of protected vitamin 10 was further purified by HPLC (6.2 mm×25 cm Zorbax-Sil column, 4 mL/min) using hexane/ethyl acetate (99.9:0.1) solvent system. Pure compound 10 was eluted at Rᵥ26 mL as a colorless oil: UV (in hexane) λₘₐₓ 224, 253, 263 nm; ¹H NMR (CDCl₃) δ0.025, 0.049, 0.066, and 0.080 (each 3H, each s, 4×SiCH₃), 0.546 (3H, s, 18-H₃), 0.565 (6H, q, J=7.9 Hz, 3×SiCH₂), 0.864 and 0.896 (9H and 9H, each s, 2×Si-t-Bu), 0.931 (3H, d, J=6.0 Hz, 21-H₃), 0.947 (9H, t, J=7.9 Hz, 3×SiCH₂CH₃), 1.188 (6H, s, 26- and 27-H₃), 2.00 (2H, m), 2.18 (1H, dd, J=12.5, 8.5 Hz, 4β-H), 2.33 (1H, dd, J=13.1, 2.9 Hz, 10β-H), 2.46 (1H, dd J=12.5, 4.5 Hz, 4α-H), 2.52 (1H, dd, J=13.1, 5.8 Hz, 10α-H), 2.82 (1H, br d, J=12 Hz, 9β-H), 4.43 (2H, m, 1β- and 3α-H), 4.92 and 4.97 (1H and 1H, each s, ═CH₂), 5.84 and 6.22 (1H and 1H, each d, J=11.0 Hz, 7- and 6-H); MS m/z (relative intensity) 758 (M+, 17), 729 (M+-Et, 6), 701 (M+-t-Bu, 4), 626 (100), 494 (23), 366 (50), 73 (92).

Protected vitamin 10 (4.3 mg) was dissolved in benzene (150 μL) and the resin (AG 50W-X4, 60 mg; prewashed with methanol) in methanol (800 μL) was added. The mixture was stirred at room temperature under argon for 17 h., diluted with ethyl acetate/ether (1:1, 4 mL) and decanted. The resin was washed with ether (8 mL) and the combined organic phases washed with brine and saturated NaHCO₃, dried (MgSO₄) and evaporated. The residue was purified by HPLC (62 mm×25 cm Zorbax-Sil column, 4 mL/min.) using hexane/2-propanol (9:1) solvent system. Analytically pure 2-methylene-19-nor-vitamin 11 (2.3 mg, 97%) was collected at Rᵥ 29 mL (1α,25-dihydroxyvitamin D₃ was eluted at Rᵥ 52 mL in the same system) as a white solid: UV (in EtOH) λₘₐₓ 243.5, 252, 262.5 nm; ¹H NMR (CDCl₃) δ0.552 (3H, s, 18-H₃), 0.941 (3H, d, J=6.4 Hz, 21-H₃), 1.222 (6H, s, 26- and 27-H₃), 2.01 (2H, m), 2.27-2.36 (2H, m), 2.58 (1H, m), 2.80-2.88 (2H, m), 4.49 (2H, m, 1β- and 3α-H), 5.10 and 5.11 (1H and 1H, each s, ═CH₂), 5.89 and 6.37 (1H and 1H, each d, J=11.3 Hz, 7- and 6-H); MS m/z (relative intensity) 416 (M+, 83), 398 (25), 384 (31), 380 (14), 351 (20), 313 (100).

### Example 2

Preparation of (20S)-1α,25-dihydroxy-2-methylene-19-nor-vitamin D₃ (15)

Scheme II illustrates the preparation of protected (20S)-25-hydroxy Grundmann's ketone 13, and its coupling with phosphine oxide 8 (obtained as described in Example 1).

(a) Silylation of Hydroxy Ketone 12

(20S)-25-[(Triethylsilyl)oxy]-des-A,B-cholestan-8-one (13). A solution of the ketone 12 (Tetrionics, Inc. Madison, Wis.; 56 mg, 0.2 mmol) and imidazole (65 mg, 0.95 mmol) in anhydrous DMF (1.2 mL) was treated with triethylsilyl chloride (95 μL, 0.56 mmol), and the mixture was stirred at room temperature under argon for 4 h. Ethyl acetate was added and water, and the organic layer was separated. The ethyl acetate layer was washed with water and brine, dried (MgSO₄) and evaporated. The residue was passed through a silica Sep-Pak cartridge in hexane/ethyl acetate (9:1) and after evaporation, purified by HPLC (9.4 mm×25 cm Zorbax-Sil column, 4 mL/min) using hexane/ethyl acetate (9:1) solvent system. Pure protected hydroxy ketone 13 (55mg, 70%) was eluted at Rᵥ 35 mL as a colorless oil: ¹H NMR (CDCl₃) δ0.566 (6H, q, J=7.9 Hz, 3×SiCH₂), 0.638 (3H, s, 18-H₃), 0.859 (3H, d, J=6.0 Hz, 21-H₃), 0.947 (9H, t, J=7.9 Hz, 3×SiCH₂CH₃), 1.196 (6H, s, 26- and 27-H₃), 2.45 (1H, dd, J=11.4, 7.5 Hz, 14α-H).

(b) Wittig-Horner Coupling of Protected (20S)-25-hydroxy Grundmann's Ketone 13 With the Phosphine Oxide 8

(20S)-1α,25-Dihydroxy-2-methylene-19-nor-vitamine D₃ (15). To a solution of phosphine oxide 8 (15.8 mg, 27.1 μmol) in anhydrous THF (200 μL) at 0° C. was slowly added n-BuLi (2.5M in hexanes, 11 μL, 27.5 μmol) under argon with stirring. The solution turned deep orange. The mixture was cooled to −78° C. and a precooled (−78° C.) solution of protected hydroxy ketone 13 (8.0 mg, 20.3 μmol) in anhydrous THF (100 μL) was slowly added. The mixture was stirred under argon at −78° C. for 1 h. and at 0C. for 18 h. Ethyl acetate was added, and the organic phase was washed with brine, dried (MgSO₄) and evaporated. The residue was dissolved in hexane and applied on a silica Sep-Pak cartridge, and washed with hexane/ethyl acetate (99.5:0.5, 20 mL) to give 19-nor-vitamin derivative 14 (7 mg, 45%) as a colorless oil. The Sep-Pak was then washed with hexane/ethyl acetate (96:4, 10 mL) to recover some unchanged C,D-ring ketone 13 (4 mg), and with ethyl acetate (10 mL) to recover diphenylphosphine oxide (9 mg). For analytical purpose a sample of protected vitamin 14 was further purified by HPLC (6.2 mm×25 cm Zorbax-Sil column, 4 mL/min) using hexane/ethyl acetate (99.9:0.1) solvent system.

14: UV (in hexane) λₘₐₓ 244, 253.5, 263 nm; ¹H NMR (CDCl₃) δ0.026, 0.049, 0.066 and 0.080 (each 3H, each s, 4×SiCH₃), 0.541 (3H, s, 18-H₃), 0.564 (6H, q, J=7.9 Hz, 3×SiCH₂), 0.848 (3H, d, J=6.5 Hz, 21-H₃), 0.864 and 0.896 (9H and 9H, each s, 2×Si-t-Bu), 0.945 (9H, t, J=7.9 Hz, 3×SiCH₂CH₃), 1.188 (6H, s, 26- and 27-H₃), 2.15-2.35 (4H, br m), 2.43-2.53 (3H, br m), 2.82 (1H, br d, J=12.9 Hz, 9β-H), 4.42 (2H, m, 1β- and 3α-H), 4.92 and 4.97 (1H and 1H, each s, ═CH₂), 5.84 and 6.22 (1H and 1H, each d, J=11.1 Hz, 7- and 6-H); MS m/z (relative intensity) 758 (M+, 33), 729 (M+-Et, 7), 701 (M+-t-Bu, 5), 626 (100), 494 (25), 366 (52), 75 (82), 73 (69).

Protected vitamin 14 (5.0 mg) was dissolved in benzene (160 μL) and the resin (AG 50W-X4, 70 mg; prewashed with methanol) in methanol (900 μL) was added. The mixture was stirred at room temperature under argon for 19 h. diluted with ethyl acetate/ether (1:1, 4 mL) and decanted. The resin was washed with ether (8 mL) and the combined organic phases washed with brine and saturated NaHCO₃, dried (MgSO₄) and evaporated. The residue was purified by HPLC (6.2 mm×25 cm Zorbax-Sil column, 4 mL/min.) using hexane/2-propanol (9:1) solvent system. Analytically pure 2-methylene-19-nor-vitamin 15 (2.6 mg, 95%) was collected at Rᵥ 28 mL [(20R)-analog was eluted at Rᵥ 29 mL and 1α,25-dihydroxyvitamin D₃ at Rᵥ 52 mL in the same system] as a white solid: UV (in EtOH) λₘₐₓ 243.5, 252.5, 262.5 nm; ³H NMR (CDCl₃) δ0.551 (3H, s, 18-H₃), 0.858 (3H, d, J=6.6 Hz, 21-H₃), 1.215 (6H, s, 26- and 27-H₃), 1.95-2.04 (2H, m), 2.27-2.35 (2H, m), 2.58 (1H, dd, J=13.3, 3.0 Hz), 2.80-2.87 (2H, m), (2H, m, 1β- and 3α-H), 5.09 and 5.11 (1H and 1H, each s, ═CH₂), 5.89 and 6.36 (1H and 1H, each d, J=11.3 Hz, 7- and 6-H); MS m/z (relative intensity) 416 (M+, 100), 398 (26), 380 (13), 366 (21), 313 (31).

Biological Activity of 2-Methylene-substituted 19-nor-1,25-(OH)₂D₃ Compounds and Their 20S-isomers

The biological activity of compounds of Formula I was set forth in U.S. Pat. No. 5,843,928 as follows. The introduction of a methylene group to the 2-position of 19-nor-1,25-(OH)₂D₃ or its 20S-isomer had little or no effect on binding to the porcine intestinal vitamin D receptor. All compounds bound equally well to the porcine receptor including the standard 1,25-(OH)₂D₃. It might be expected from these results that all of the compounds would have equivalent biological activity. Surprisingly, however, the 2-methylene substitutions produced highly selective analogs with their primary action on bone. When given for 7 days in a chronic mode, the most potent compound tested was the 2-methylene-19-nor-20S-1,25-(OH)₂D₃ (Table 1). When given at 130 pmol/day, its activity on bone calcium mobilization (serum calcium) was of the order of at least 10 and possible 100-1,000 times more than that of the native hormone. Under identical conditions, twice the dose of 1,25-(OH)₂D₃ gave a serum calcium value of 13.8 mg/100 ml of serum calcium at the 130 pmol dose. When given at 260 pmol/day, it produced the astounding value of 14 mg/100 ml of serum calcium at the expense of bone. To show its selectivity, this compound produced no significant change in intestinal calcium transport at either the 130 or 260 pmol dose, while 1,25-(OH)₂D₃ produced the expected elevation of intestinal calcium transport at the only dose tested, i.e. 260 pmol/day. The 2-methylene-19-nor-1,25-(OH)₂D₃ also had extremely strong bone calcium mobilization at both dose levels but also showed no intestinal calcium transport activity. The bone calcium mobilization activity of this compound is likely to be 10-100 times that of 1,25-(OH)₂D₃. These results illustrate that the 2-methylene and the 20S-2-methylene derivatives of 19-nor-1,25-(OH)₂D₃ are selective for the mobilization of calcium from bone. Table 2 illustrates the response of both intestine and serum calcium to a single large dose of the various compounds; again, supporting the conclusions derived from Table 1.

The results illustrate that 2-methylene-19-nor-20S-1,25-(OH)₂D₃ is extremely potent in inducing differentiation of HL-60 cells to the monocyte. The 2-methylene-19-nor compound had activity similar to 1,25-(OH)₂D₃. These results illustrate the potential of the 2-methylene-19-nor-20S-1,25-(OH)₂D₃and 2-methylene-19-nor-1,25-(OH)₂D₃ compounds as anti-cancer agents, especially against leukemia, colon cancer, breast cancer and prostate cancer, or as agents in the treatment of psoriasis.

Competitive binding of the analogs to the porcine intestinal receptor was carried out by the method described by Dame et al. (Biochemistry 25, 4523-4534, 1986).

The differentiation of HL-60 promyelocytic into monocytes was determined as described by Ostrem et al (J. Biol. Chem. 262, 14164-14171, 1987).

TABLE 1
Response of Intestinal Calcium Transport and Serum Calcium (Bone
Calcium Mobilization) Activity to Chronic Doses of
2-Methylene Derivatives of 19-Nor-1,25-(OH)₂D₃ and its 20S Isomers

| | Dose | Intestinal Calcium | Serum |
|---|---|---|---|
| | (pmol/day/ | Transport | Calcium |
| Group | 7 days) | (S/M) | (mg/100 ml) |
| Vitamin D Deficient | Vehicle | 5.5 ± 0.2 | 5.1 ± 0.16 |
| 1,25-(OH)₂D₃ Treated | 260 | 6.2 ± 0.4 | 7.2 ± 0.5 |
| 2-Methylene-19-Nor-1,25- | 130 | 5.3 ± 0.4 | 9.9 ± 0.2 |
| (OH)₂D₃ | 260 | 4.9 ± 0.6 | 9.6 ± 0.3 |
| 2-Methylene-19-Nor-20S- | 130 | 5.7 ± 0.8 | 13.8 ± 0.5 |
| 1,25-(OH)₂D₃ | 260 | 4.6 ± 0.7 | 14.4 ± 0.6 |

Male weanling rats were obtained from Sprague Dawley Co. (Indianapolis, Ind.) and fed a 0.47% calcium, 0.3% phosphorus vitamin D-deficient diet for 1 week and then given the same diet containing 0.02% calcium, 0.3% phosphorus for 2 weeks. During the last week they were given the indicated dose of compound by intraperitoneal injection in 0.1 ml 95% propylene glycol and 5% ethanol each day for 7 days. The control animals received only the 0.1 ml of 95% propylene glycol, 5% ethanol. Twenty-four hours after the last dose, the rats were sacrificed and intestinal calcium transport was determined by everted sac technique as previously described and serum calcium determined by atomic absorption spectrometry on a model 3110 Perkin Elmer instrument (Norwalk, Conn.). There were 5 rats per group and the values represent mean (±)SEM.

TABLE 2
Response of Intestinal Calcium Transport and Serum Calcium (Bone
Calcium Mobilization) Activity to Chronic Doses of
2-Methylene Derivatives of 19-Nor-1,25-(OH)₂D₃ and its 20S Isomers

| | Intestinal Calcium | Serum |
|---|---|---|
| | Transport | Calcium |
| Group | (S/M) | (mg/100 ml) |
| -D Control | 4.2 ± 0.3 | 4.7 ± 0.1 |
| 1,25-(OH)₂D₃ | 5.8 ± 0.3 | 5.7 ± 0.2 |
| 2-Methylene-19-Nor-1,25-(OH)₂D₃ | 5.3 ± 0.5 | 6.4 ± 0.1 |
| 2-Methylene-19-Nor-20S-1,25-(OH)₂D₃ | 5.5 ± 0.6 | 8.0 ± 0.1 |

Male Holtzman strain weanling rats were obtained from the Sprague Dawley Co. (Indianapolis, Ind.) and fed the 0.47% calcium, 0.3% phosphorus diet described by Suda et al. (J. Nutr. 100, 1049-1052, 1970) for 1 week and then fed the same diet containing 0.02% calcium and 0.3% phosphorus for 2 additional weeks. At this point, they received a single intrajugular injection of the indicated dose dissolved in 0.1 ml of 95% propylene glycol/5% ethanol. Twenty-four hours later they were sacrificed and intestinal calcium transport and serum calcium were determined as described in Table 1. The dose of the compounds was 650 pmol and there were 5 animals per group. The data are expressed as mean (±)SEM.

Accordingly, compounds of the following formulae Ia, are along with those of formula I, also encompassed by the present invention:

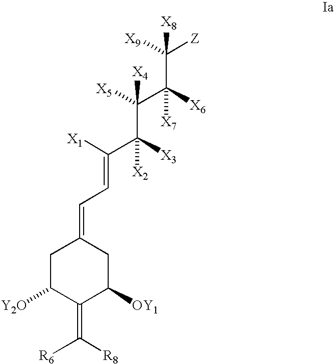

In the above formula Ia, the definitions of Y₁, Y₂, R₆, R₈ and Z are as previously set forth herein. With respect to X₁, X₂, X₃, X₄, X₅, X₆, X₇, X₈ and X₉, these substituents may be the same or different and are selected from hydrogen or lower alkyl, i.e., a C₁₋₅ alkyl such as a methyl, ethyl or n-propyl. In addition, paired substituents X₁ and X₄or X₅, X₂ or X₃ and X₆ or X₇, X₄ or X₅ and X₈ or X₉, together with the three adjacent carbon atoms of the central part of the compound, which correspond to positions 8, 14, 13 or 14, 13, 17 or 13, 17, 20 respectively, can be the same or different and form a saturated or unsaturated, substituted or unsubstituted, carbocyclic 3, 4, 5, 6 or 7 membered ring.

Preferred compounds of the present invention may be represented by one of the following formulae:

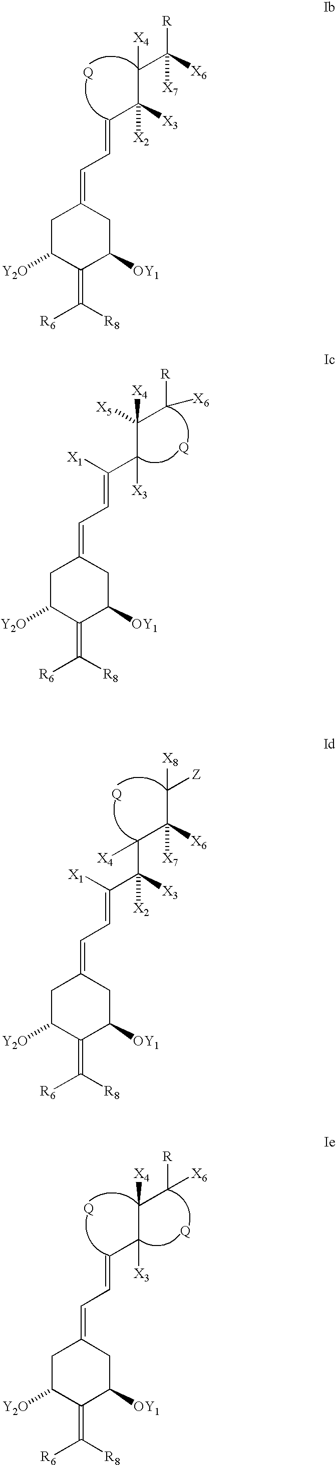

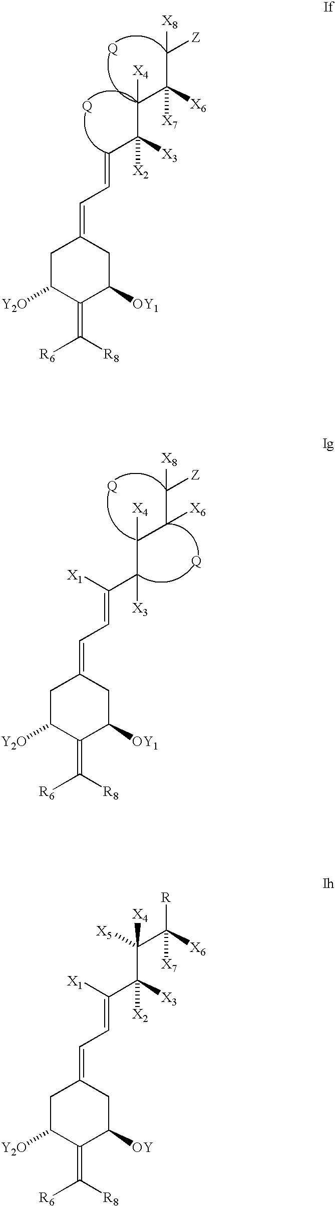

In the above formulae Ib, Ic, Id, Ie, If, Ig and Ih, the definitions of Y₁, Y₂, R₆, R₈, R, Z, X₁, X₂, X₃, X₄, X₅, X₆, X₇, and X₈ are as previously set forth herein. The substituent Q represents a saturated or unsaturated, substituted or unsubstituted, hydrocarbon chain comprised of 0, 1, 2, 3 or 4 carbon atoms, but is preferably the group —(CH₂)ₖ— where k is an integer equal to 2 or 3.

Methods for making compounds of formulae Ia-Ih are known. Specifically, reference is made to International Application Number PCT/EP94/02294 filed Jul. 7, 1994, and published Jan. 19, 1995, under International Publication Number WO95/01960.

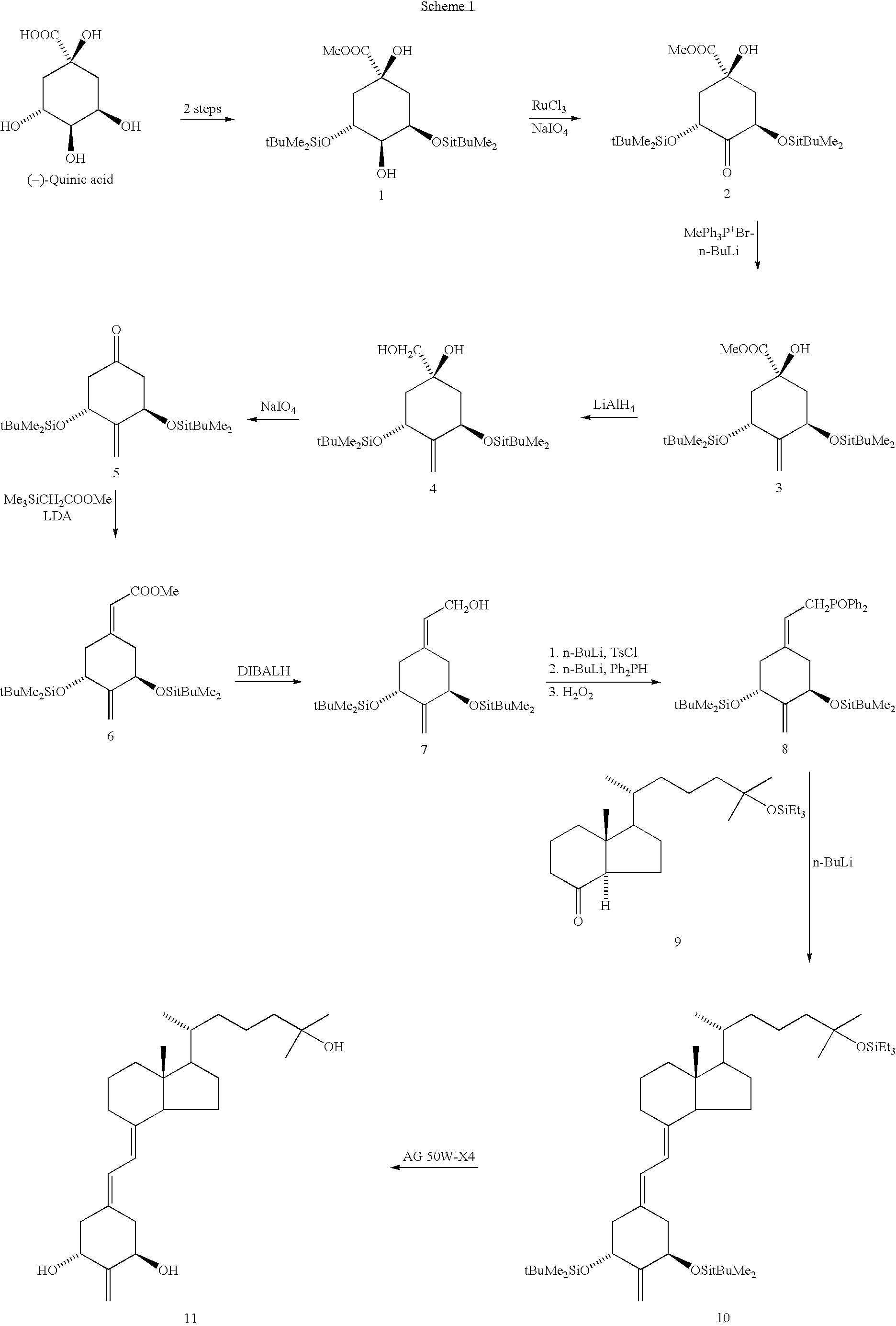

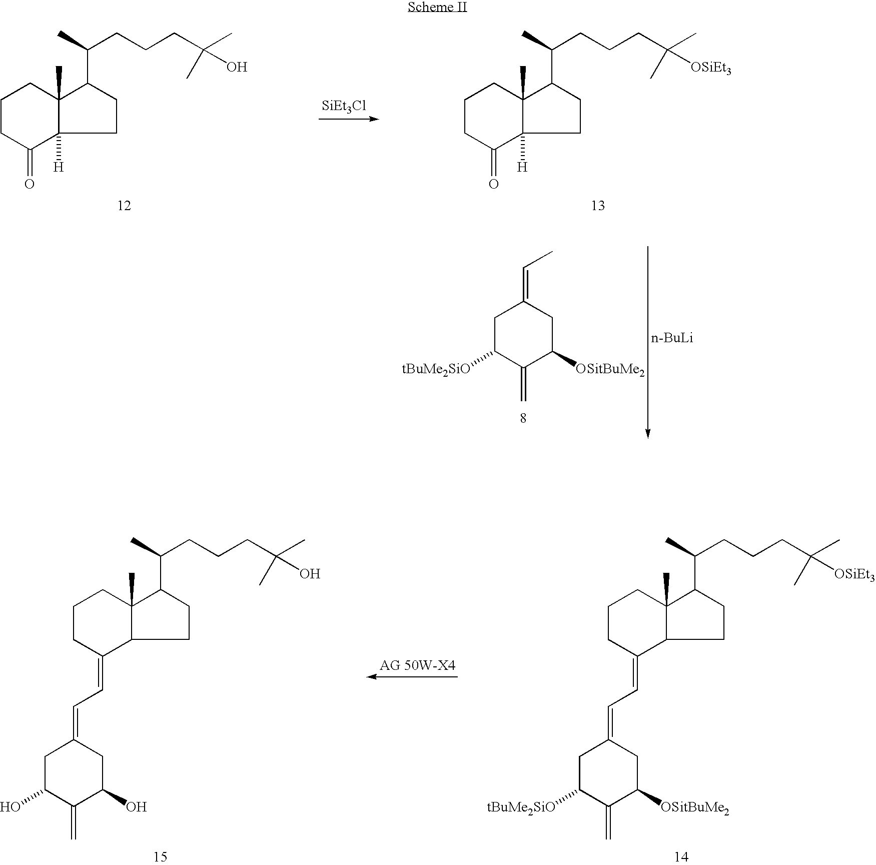

1. A method of treating anorexia or low bone mass in females exhibiting aggressive athletic behavior comprising administering to a patient in need thereof a therapeutically effective amount of 2-methylene-19-nor-20(S)-1α,25-dihydroxyvitamin D₃.

2. The method of claim 1 wherein the 2-methylene-19-nor-20(S)-1α,25-dihydroxyvitamin D₃ is administered orally.

3. The method of claim 1 wherein the 2-methylene-19-nor-20(S)-1α,25-dihydroxyvitamin D₃ is administered parenterally.

4. The method of claim 1 wherein the 2-methylene-19-nor-20(S)-1α,25-dihydroxyvitamin D₃ is administered transdermally.

5. The method of claim 1 wherein anorexia is treated.

6. The method of claim 1 wherein low bone mass in females exhibiting aggressive athletic behavior is treated.
